(19) United States
(12) Patent Application Publication
(10) Pub. No.: US 2004/0249265 A1
(43) Pub. Date: Dec. 9, 2004

(54) Method and apparatus for examining blood vessel responsiveness

(76) Inventors: Friedrich Fuchs, Rottenbach (DE); Rainer Kuth, Herzogenaurach (DE); Christine Lorenz, Frederick, MD (US)

Correspondence Address: SCHIFF HARDIN, LLP, PATENT DEPARTMENT, 6600 SEARS TOWER, CHICAGO, IL, 60606-6473, US

(21) Appl. No.: 10/815,042

(22) Filed: Mar. 31, 2004

(30) Foreign Application Priority Data
Mar. 31, 2003 (DE) 103 14 535.4

(51) Int. Cl. A61B 5/05

(52) U.S. Cl. 600/419

(57) ABSTRACT

In a device and method for examining the function, in particular the responsiveness, of blood vessels in a patient by creating a vascular constriction or dilation, the body part to be examined is heated or cooled in a controlled manner with an arrangement for local heating or cooling, and the blood or body-fluid flow through vessels that are dilated or constricted is measured dependent on the heating or cooling in this part of the body.

### BACKGROUND OF THE INVENTION

1. Field of the Invention

The present invention concerns a procedure for examining the function, in particular the responsiveness, of blood vessels in a patient to a vascular constriction or dilation.

2. Description of the Prior Art

For many clinical problems regarding vascular diseases, in particular concerning peripheral vessels, an examination procedure is needed that enables measurement of the responsiveness of the musculature vessels and the interior vascular wall, which enables the constriction or dilation of the blood vessels. Especially preferable are procedures that are not invasive, require no medical Indication, and can also be performed by non-medical personnel.

One possible area of application for the suggested procedure and device is the diagnosis of arterial circulatory disorder. This disease is manifested by severe calf pain after walking along a certain path, which makes a person stop, and disappears again after a few minutes due to sufficient circulation to the musculature during periods of rest. The pain reappears upon renewed stress. Arterial occlusive diseases of the legs are mostly the cause of this disease.

Medications are already known that lead to a short-term and temporary constriction or dilation of the vessels, e.g. nitropreparations, but these have side effects and require a strict medical indication of their use. Moreover, they can only be administered by a physician and require monitoring, such as the measurement of blood pressure and pulse rate. An additional disadvantage Is that no alternating stress is possible during an examination, e.g. it would be desirable to dilate, constrict, re-dilate, etc., the vessels.

### SUMMARY OF THE INVENTION

An object of the present invention is to provide a procedure for examining the function of blood vessels that allows for a reliable diagnosis and that can also be performed by non-medical personnel.

This object is achieved in accordance with the invention with the invention in a method wherein the body part to be examined is warmed or cooled in a controlled manner (i.e., a controlled localized temperature change is effected) with an arrangement for local heating or an arrangement for local cooling, and the blood or body fluid flow through vessels that are dilated or constricted is measured dependent on the heating or cooling in this part of the body.

In the inventive procedure, a reliable measurement of the responsiveness of blood vessels can be made by the behavior of the body parts in question being examined under a cold or heat stimulus. The heating of blood vessels generally leads to a dilation, which results in a corresponding change in the flow. A cooling causes a contraction of the vessels, which also can be measured. An advantage of the inventive procedure is that no physician is required for the application, which leads to lower examination costs.

In an embodiment of the invention, a source of radiation, in particular a radiant heater, is used as the arrangement for heating the part of the body to be examined. The temperature of the body part to be examined, e.g. the surface of the skin, preferably is measured during the performance of the procedure, so that a relationship between the temperature change and the change in the fluid flow can be deduced.

In a further embodiment of the inventive procedure, a Peltier element is used for warming or cooling. An electric current is used to heat or cool with this version.

The inventive procedure can be performed particularly safely and reliably by placing a temperable compress on the body part to be examined, as the arrangement for heating or cooling.

The compress can be brought to a specific temperature so that the body part to be examined attains this temperature, at least on its surface, within a short period of time.

In a further embodiment of the invention, a fluid able to absorb or emit heat flows through the compress. Preferably, a gas or a liquid is used as the fluid. The use of such a liquid gives a particularly uniform and quick transfer of heat.

It is also possible for the fluid to be stored in a storage container connected to the compress in the inventive procedure. Thus, it can be ensured that the fluid has a homogenous temperature so that the examination can be particularly precise. It is particularly convenient in the inventive procedure to use a heating and/or cooling device to set the fluid temperature. In this case, trained medical personnel can set the desired fluid temperature, which can lie above or below body temperature or can be equal to body temperature

In a further embodiment of the invention, at least two storage containers are used respectively for fluid at different temperatures. The blood vessel then can be subjected to alternating stress with a cold fluid and a warm fluid, so that the behavior of the vessels to be constricted and dilated can be examined. Thus, it is particularly convenient for the compress placed on the body part to be examined to be connected selectively with one of the storage containers at a time via a valve. If the user needs to switch between the warm fluid and the cold fluid, the valve Is used so that the fluid can be taken from the appropriate storage container.

In a further embodiment of the procedure the fluid is conveyed via a return line in a circulation loop between the compress and the storage container(s). This minimizes fluid use, since the fluid is circulated; and the heated or cooled fluid can be used further so that the energy used for cooling or heating is minimal.

It is especially advantageous to use water or oil as the fluid in the inventive procedure. The use of oil is advantageous if the patent is to be examined using magnetic resonance tomography, since in this case the field of view can be limited to the actual body part. Oil can be seen in an MR image, because it emits an MR signal. Normally this would be bothersome, but it can be desired if one wants to measure the temperature with MR over the variation of the relaxation times, e.g. for monitoring.

Alternatively, a liquid that is inert for magnetic resonance tomography can be used as the fluid Preferably, such a fluid is a fluorinated carbon compound that is non-insulating and non-ferromagnetic and that has no unbalanced protons.

In another embodiment of the inventive procedure the temperature of the fluid is controlled by a controller for the heating and/or cooling device. This controller can be contained, for example, in a housing of the heating and/or cooling device, or it can be part of an external device, such as a PC.

In a further embodiment of the invention, the fluid temperature is measured on the body part to be examined using a temperature sensor. This temperature sensor can be arranged within the compress that is placed on the body part to be examined. Alternatively, the temperature sensor can be placed on the outside of the compress.

Particularly favorable results can be achieved In an embodiment of the inventive procedure wherein the compress with fluid flowing through it is coupled with an external device, in particular a magnetic resonance tomography device or an X-ray computed tomography device. This combination enables data exchange between the device with the fluid flowing through it and the external device, so that the temperature data from a specific period in time can be assigned to the examination data. Thus, the temperature distribution is obtained as well as the provoked reaction of the vessels of the patient as functions of time. It is therefore convenient for the temperature distribution of the fluid also to be recorded as a function of time.

In order to further simplify the use of the inventive procedure, a material compatible with magnetic resonance tomography, in particular polytetrafluomethylene, can be used for the compress. This material causes no disturbances in the examination image.

In a further embodiment several compresses, placed on several sides of the body part to be examined, can be used in the procedure. For certain body parts, it makes sense to use a pressure cuff as the compress, e.g. when examining the legs.

With the procedure based on the invention, it is especially preferred for the liquid flow to be measured using magnetic resonance tomography. Thus, the liquid flow can be measured in axial sections of the body part to be examined. It is also possible to measure the liquid flow with time-of-flight MR angiography. Alternatively, the liquid flow can be measured with phase-contrast angiography.

Especially if magnetic resonance tomography is used in the inventive procedure a contrast agent can be administered to the patient before the liquid flow is measured.

In the inventive procedure, the liquid flow also can be measured using X-ray computed tomography or using digital subtraction angiography as an alternative to the aforementioned embodiments.

In order to be able to perform a diagnosis of the behavior, in particular the responsiveness, of the vessel, in accordance with the invention, the cross-sectional area of the vessel is calculated from the measured liquid flow.

The invention also concerns a device for controlling the temperature of a body part in order to examine the function, in particular the responsiveness, of blood vessels in a patient by creating a vascular constriction or dilation in accordance with the above-described inventive procedure.

The inventive device can include a compress placed on the body part to be examined and containing flowing fluid and an arrangement for setting the fluid temperature,

### DESCRIPTION OF THE DRAWINGS

FIG. 1 is a schematic diagram of an embodiment of the inventive device with two storage containers for fluid and an examination cuff for a patient, who is examined using magnetic resonance tomography.

FIG. 2 is a schematic diagram of measured values of the liquid flow and the blood vessel diameter dependent an the temperature, obtained in accordance with the invention.

### DESCRIPTION OF THE PREFERRED EMBODIMENTS

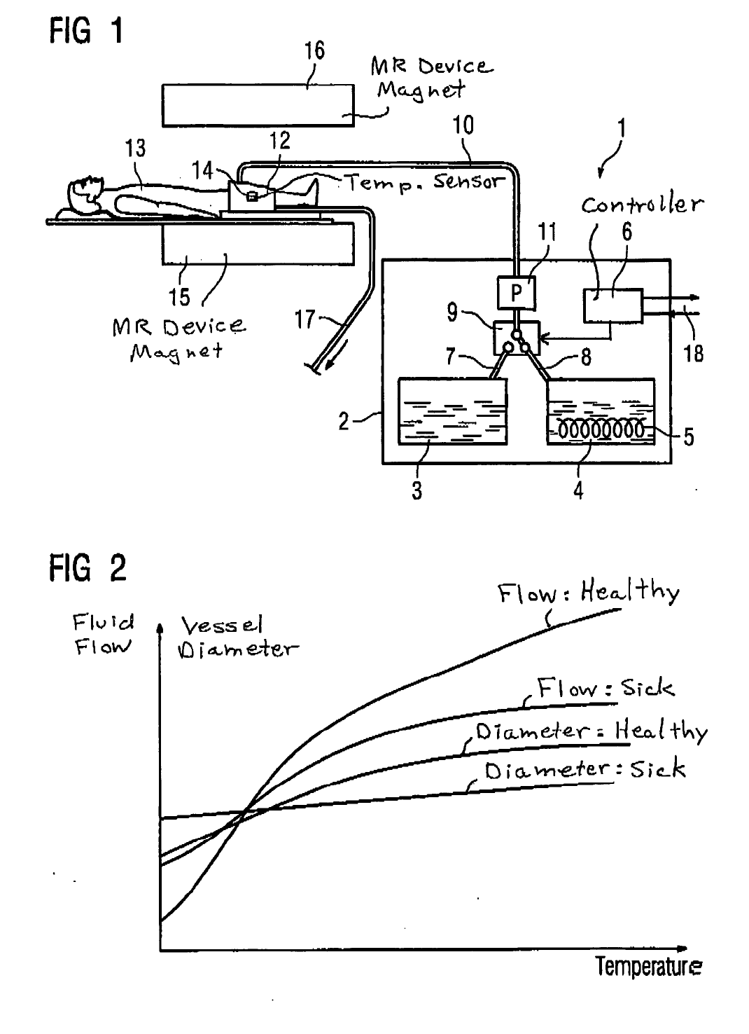

The device 1 shown in FIG. 1 has a housing 2, in which a first storage container 3 and a second storage container 4 accommodate a fluid, The storage device 3 is filled with cold water; it can also be directly connected to a water tap. The storage device 4 is intended for heated fluids; thus, it has a heating element 5 inside, with which the fluid is heated. Moreover, temperature sensors or thermostats that are not shown in FIG. 1 and that are connected with a controller 6 in the housing 2 of the device 1 are arranged in the storage containers 3, 4,

Lines 7, 8 lead from the storage devices 3, 4, respectively, to a valve block 9, which selectively connects one of the lines 7, 8 with a supply line 10. The valve block 9 is actuated by the controller 6, which connects either the storage container 3 or the storage container 4 with the supply line 10, so that the fluid is conveyed using a pump 11 arranged within the housing 2.

The supply line 10 at least has a fluid tube, but can be designed as a tube package with additional lines e.g. for transferring sensor signals. The end of the supply line 10 is connected to a compress in the form of a cuff 12, which goes around the leg of a patient 13. The cuff 12 in this embodiment is formed of a material compatible with magnetic resonance tomography, such as polytetrafluoroethylene, for example. A detector for temperature is arranged inside the cuff 12. In the simplest case, this can be an electrical temperature sensor 14, however, it is also possible to measure the surface temperature in a contact-free manner in order to avoid interfering with the MR sequence.

During the examination, the patient 13 lies on a table of the MR tomography device, which is represented schematically in FIG. 1 by the indicated magnets 15, 16. This is a conventional magnetic resonance tomography system, also called a magnetic resonance imaging system, and can be used for a wide variety of examinations.

An outflow line 17 is attached to the cuff 12, via which the fluid is fed to a drain, in particular when water is used as the fluid. If an oil or a fluorinated carbon compound is used, however, the fluid is collected for reuse or fed back into the appropriate storage container 3 or 4, in which it is either reheated or re-cooled as needed.

The cuff 12 is designed so that fluid flows through almost the entire interior thereof so that the cuff 12 can be maintained at a constant temperature and this temperature is transferred to the body part of the patient 13 to be examined. The surface temperature of the cuff 12 or the skin of the patient 13 is thereby recorded with the temperature sensor 14, the measured value of which is transmitted via a line (not shown in FIG. 1) or wirelessly to the controller 6. During the examination, the current temperature is continuously ascertained via the temperature sensor 14 and saved by the controller 6. The controller 6 can supply the measured value to an external device via a data line 18. For example, this can be a PC or the operating unit of an MR device or a similar device.

In the depicted embodiment, an MR-inert liquid is used in the examination; that is, it is non-conducting, non-ferromagnetic, and has no unbalanced protons. A fluorinated carbon is particularly suitable for this purpose.

The examination begins with a heating of the leg of the patient 13 to be examined. The fluid in the storage container 4 is brought to the increased temperature by the heating element 5. The valve block 9 is located in the position indicated in FIG. 1 and the pump 11 pumps the fluid through the supply line 10 to the cuff 12, which then is heated, which leads to a warming of the leg of the patient 13. In this state, an image of the body part to be examined and its vessels is generated using magnetic resonance tomography. Several images are obtained, which are shifted from image-to-image in the axial direction. The diameter of one or more vessels can be ascertained from this captured image data.

With this known diameter, the liquid flow oan be calculated as volume flow according to the Hagen-Poiseuille equation. In addition to the diameter of the vessels, the pressure difference Ap between the two examined sections as well as the distance between the two sections must be known. The liquid flow can be measured with MR without a contrast medium. e.g., with time-of-flight MR angiography or with phase contrast angiography.

As an alternative, the liquid flow can be measured with MR after the administration of a contrast agent.

Several contrast agent administrations can be made one after the other, if the body part to be examined is in thermal equilibrium. The absolute or relative cross-sectional area or the diameter or radius of one or more blood vessels is calculated from the different flows. The liquid flow is proportional to the fourth power of the diameter or the radius of the vessel. The flow in peripheral vessels is always laminar so that the aforementioned Hagen-Poiseuille equation may be used.

In order to subject the blood vessel to be examined to alternating stress, it is cooled after this first measurement. For this, an appropriate switch signal Is given from the controller 6 to the valve block 9, which then connects the storage container 3 with the cold fluid to the supply line 10. The cold fluid subsequently is pumped through the line 10 and the cuff 12 so that the body part of the patient 13 to be examined is cooled. Once thermal equilibrium is achieved, an MR measurement is then performed. Then, a third examination can be performed with yet another changed temperature. It is also possible to set the speed of the reaction of the blood vessel in relation to the temperature in order to obtain information on the condition of the blood vessels.

Various standard examination programs that run automatically and require no intervention from operating personnel are stored in the controller 6, but it is also possible to enter examination programs manually. The evaluation of the data can be transferred to a calculating unit of the device 1. It is also possible to transfer the data via the data line 18 to a PC or to the MR system in order to perform a correlation with the MR data if necessary and to evaluate the examination data. Generally, however examination findings can be derived from the obtained MR data alone by comparing at least two measurements with each other at different temperatures.

A reproducible and highly accurate analysis of the functional efficiency of blood vessels without contrast media and without administering additional medication is achieved with the inventive device 1 and the inventive procedure. The constriction and dilation of vessels under the effect of temperature can be measured directly by taking the diameter of the vessels from the images. This task is performed manually or automatically via a software program. Alternatively or additionally, the diameter and the change in the diameter can be determined from the liquid flow.

FIG. 2 show examples for measured values of the liquid flow and the vessel diameter depending on the temperature of the respective vessel. The curve progressions are schematic representations and show the corresponding measured values of a healthy and a sick patient.

The lower curve shows the vessel diameter of a sick patient, The diameter increases only slightly with an increase in the temperature, which indicates a disease of the vessels. In contrast, in the curve above this one, a greater increase in the vessel diameter is seen between the lower temperature and the increased temperature, which leads us to conclude that the vessels are healthy.

Both of the upper curves show the liquid flow of a sick and a healthy patient depending on the temperature. It can be seen that the liquid flow of the sick patient lies predominantly below that of a healthy patient.

Generally, one of the two curves is already sufficient for diagnosis, since the liquid flow and the blood vessel diameter are linked via the Hagen-Poiseuille equation.

Although modifications and changes may be suggested by those skilled in the art, it is the intention of the Inventors to embody within the patent warranted hereon all changes and modifications as reasonably and properly come within the scope of their contribution to the art,

We claim as our invention:

1. A method for examining responsivity of blood vessels In a patient to a vascular constriction or dilation, comprising the steps of:
producing a localized, controlled temperature change in a body part of a living subject containing body fluid vessels in which a body fluid flow exists and selectively dilating or constricting said vessels; and
measuring said body fluid flow in said vessels after said temperature change dependent on said temperature change.

2. A method as claimed in claim 1 wherein the step of producing a localized, controlled temperature change comprises radiantly heating said body part.

3. A method as claimed in claim 1 comprising producing said vocalized, controlled temperature change with a Peltier element that interacts with said body part.

4. A method as claimed in claim 1 wherein the step of producing a controlled, localized temperature change comprises applying a temperature-adjustable compress to said body part.

5. A method as claimed in claim 4 comprising adjusting a temperature of said compress by flowing fluid through said compress at a selected temperature.

6. A method as claimed in claim 5 comprising storing said fluid in a container attached to said compress.

7. A method as claimed in claim 5 comprising setting said fluid at said selected temperature with a temperature-setting device, selected from the group consisting of heating devices and cooling devices, that Interact with said fluid.

8. A method as claimed in claim 5 comprising storing said fluid In different, separate storage containers, and maintaining the fluid respectively stored in said storage containers at different temperatures.

9. A method as claimed in claim 8 comprising selectively causing fluid from one of said storage containers at a time to flow through said compress via a valve connected between said two storage containers and said compress.

10. A method as claimed in claim 5 comprising storing said fluid in a storage container to said compress via feed line and a return line, and circulating said fluid between said compress and said container via said feed line and said return line.

11. A method as claimed in claim 5 comprising employing a fluid selected from the group consisting of water and oil as said fluid in said compress.

12. A method as claimed in claim 5 comprising employing a fluid that is inert for magnetic resonance tomography as said fluid in said compress.

13. A method as claimed in claim 5 comprising employing fluorinated carbon as said fluid in said compress.

14. A method as claimed in claim 5 comprising regulating the temperature of said fluid with a regulating device that interacts with said fluid, selected from the group consisting of heating regulators and cooling regulators.

15. A method as claimed in claim 6 comprising measuring a temperature of the body part using a temperature sensor.

16. A method as claimed in claim 15 comprising measuring said temperature over time for obtaining a temperature curve with respect to time.

17. A method as claimed in claim 6 comprising controlling flow of said fluid through said compress with an external imaging device in which said body part is disposed, selected from the group consisting of magnetic resonance tomography devices and x-ray computed tomography devices.

18. A method as claimed in claim 5 comprising forming said compress of a material that is compatible for magnetic resonance tomography.

19. A method as claimed in claim 18 comprising forming said compress of polytetrafluoroethylene.

20. A method as claimed in claim 5 comprising employing a cuff as said compress.

21. A method as claimed in claim 1 wherein the step of producing a controlled, localized temperature change comprises applying a plurality of compresses respectively at different sides of said body part and flowing a temperature-adjustable fluid through said plurality of compresses.

22. A method as claimed in claim 1 comprising measuring said fluid flow in said body part using magnetic resonance tomography.

23. A method as claimed in claim 1 comprising measuring said fluid flow in axial sections of said body part.

24. A method as claimed in claim 1 comprising measuring said fluid flow in said body part using time-of-flight magnetic resonance angiography.

25. A method as claimed in claim 1 comprising measuring said fluid flow in said body part using phase contract angiography.

26. A method as claimed in claim 1 comprising administering a contrast agent to a patient possessing said body part before measuring said fluid flow in said body part, and measuring said fluid flow in said body part using an imaging modality with which said contrast agent is detectable.

27. A method as claimed in claim 1 comprising measuring said fluid flow in said body part using x-ray computed tomography.

28. A method as claimed in claim 1 comprising measuring said fluid flow in said body part using digital subtraction angiography.

29. A method as claimed in claim 1 comprising, from the measurement of said fluid flow in said body part, calculating a cross-sectional area of a blood vessel in said body part in which said fluid flows.

30. An arrangement for examining responsivity of fluid-carrying vessels in a body part of a patient by producing a vascular constriction or dilation of said vessels, comprising:
a compress adapted to be placed on a body part containing vessels in which a body fluid flows, said compress having a temperature-adjustable fluid flowing therein;
a setting device disposed in interactive relation with said fluid in said compress for setting a temperature of said fluid in said compress to cause a temperature change in said body part for constricting or dilating said vessels; and
a measuring device for measuring flow of said body fluid in said vessels in said body part after said temperature change, dependent on said temperature change.

31. An arrangement as claimed in claim 30 comprising a storage container for said fluid flowing through said compress, connected to said compress via a feed line.

32. An arrangement as claimed in claim 30 wherein said setting device is selected from the group consisting of heating devices and cooling devices.

33. An arrangement as claimed in claim 32 comprising a storage container for said fluid in said compress connected to said compress via a feed line, and wherein said setting device Interacts with said fluid in said container, and is selected from the group consisting of heating devices and cooling devices.

34. An arrangement as claimed in claim 30 wherein said setting device is a Peltier element.

35. An arrangement as claimed in claim 30 comprising a first storage container for said fluid in said compress and a second storage container for said fluid in said compress, said first and second storage containers being connected to said compress via respective feed lines, and wherein said setting device sets a temperature of the fluid in said first storage container to be different from a temperature of the fluid in said second storage container.

36. An arrangement as claimed in claim 35 comprising a valve connected to said respective feed lines of said first and second storage containers, for selectively causing fluid from one of said first and second storage containers at a time to flow in said compress.

37. An arrangement as claimed in claim 30 comprising a storage container for said fluid in said compress, connected to said compress via a feed line and a return line for circulating said fluid between said storage container and said compress.

38. An arrangement as claimed in claim 30 wherein said fluid in said compress is selected from the group consisting of water and oil.

39. An arrangement as claimed in claim 30 wherein said fluid in said compress is comprised of a material that is inert for magnetic resonance tomography.

40. An arrangement as claimed in claim 39 wherein said fluid is a fluorinated carbon compound.

41. An arrangement as claimed In claim 30 wherein said setting device comprises a controller for alternatingly heating and cooling said fluid in said compress, for alternatingly dilating and constricting said vessels in said body part.

42. An arrangement as claimed in claim 41 comprising a temperature sensor disposed at said compress, and adapted to interact with said body part, for recording a temperature of said body part.

43. An arrangement as claimed in claim 42 wherein said controller comprises a memory for storing a plurality of recorded temperatures of said body part over time as a temperature curve with respect to time.

44. An arrangement as claimed in claim 30 wherein said measurement device is selected from group consisting of magnetic resonance tomography systems and x-ray computed tomography systems.

45. An arrangement as claimed in claim 30 wherein said compress is composed of a material that is compatible with magnetic resonance tomography.

46. An arrangement as claimed in claim 45 wherein said material is polytetrafluoroethylene.

47. An arrangement as claimed in claim 30 wherein said compress is a first compress, and comprising at least one further compress, said first compress and said at least one further compress being adapted for placement at respectively different sides of said body part.

48. An arrangement as claimed in claim 30 wherein said compress is a cuff.
